(19) United States
(12) Patent Application Publication
(10) Pub. No.: US 2023/0162823 A1
(43) Pub. Date: May 25, 2023

(54) RETROACTIVE CODING FOR HEALTHCARE

(71) Applicant: Merative US L.P., Ann Arbor, MI (US)

(72) Inventors: Paul R. Bastide, Ashland, MA (US); Robert E. Loredo, North Miami Beach, FL (US); Matthew E. Broomhall, Goffstown, NH (US); Fang Lu, Billerica, MA (US)

(21) Appl. No.: 17/455,924

(22) Filed: Nov. 22, 2021

(51) Int. Cl. G16H 10/60

(52) U.S. Cl. CPC G16H 10/60

(57) ABSTRACT

A method, a computer program product, and a computer system retroactively update a record with a new code. The method includes determining the new code that is introduced into a medical coding system. The method includes determining a first patient having a first patient record including the new code. The method includes determining a correlation between the new code and an existing code. The existing code was available prior to the new code being introduced. The method includes determining a second patient having a second patient record including the existing code. The method includes determining whether the second patient is a candidate to have the second patient record retroactively updated with the new code based on a similarity analysis with the first patient. As a result of the second patient being confirmed the candidate, the method includes updating the second patient record with the new code.

### BACKGROUND

The exemplary embodiments relate generally to healthcare codes, and more particularly to determining when and how to retroactively update a record with a new code.

In the field of healthcare and life sciences, companies generate large amounts of clinical and commercial data. For example, payers, providers, pharmaceutical companies, and data scientists may use the data in analytics to derive insights that may be identified and de-identified for cohorts and patients. To manage this substantially large amount of data, an organization may offer a suite of common and flexible platform services for health data and analytics in a multi-cloud and hybrid-cloud environment. The services may include Fast Healthcare Interoperatbility Resources (FHIR), de-identification, patient insights, patient summary, precision cohorts, annotator for clinical data, insights for medical literature, etc.

The services to manage medical data may utilize a code system in which a condition, disease, or other medically related issue may be represented with a code. With an emerging disease (e.g., COVID-19), the code system and the coders generating the codes for the code systems have had to update the approach to coding and subsequently billing as well as other services for the rendered healthcare as the emerging disease is assigned a new code. With the changes from high level organizations such as the Centers for Medicare and Medicaid (CMS), new healthcare common procedure coding system (HCPCS) codes for the emerging disease (e.g., two new HCPCS codes to report COVID-19 testing) and additional codes for the emerging disease collection (e.g., two additional codes for the COVID-19 collection) are introduced which further complicate the issue of managing the medical data. The American Medical Association (AMA) also releases codes to report related aspects (e.g., antibody testing for COVID-19). These codes change or update frequently (e.g., on a daily basis) to best track, bill, and account for costs and billing parties.

In light of the dynamic nature of utilizing codes for medical data and the use of the codes in various medically related services (e.g., maintaining medical charts) and associated services (e.g., billing), conventional approaches have configured ways of tracking the medical data.

U.S. Publ. Appln. No. 2021/0142875 introduces a conventional approach where a treatment plan based on changing codes is built to a fixed set of diagnoses and ascribes a billing sequence based on the diagnoses and treatments in the plan. However, this conventional approach focuses on the association between a diagnosis in real time and the associated billing codes in a remote setting. In its entirety, this conventional approach is not configured to retroactively apply changes to codes.

U.S. Publ. Appln. No. 2017/0235891 introduces another conventional approach where a natural language processing engine extracts concepts and processes Z-segments (e.g., doctor's notes) such that insights may be derived from the content. However, this conventional approach only provides a mechanism by which concepts are extracted without any relation to utilizing codes for medical data.

U.S. Publ. Appln. No. 20180081859 introduces a further conventional approach where concepts and facts are extracted from text and is used to train a model that associates the text with medical codes. As one skilled in the art will understand, this further conventional approach is typically used in Medical Speed-To-Text. However, this conventional approach simply utilizes whatever codes are available with no mechanism for retroactive coding.

U.S. Publ. Appln. No. 2015/0046181 introduces yet another conventional approach where historic claims data is used to classify current claims data as fraud or not-fraud. This conventional approach utilizes classification specific mechanisms. However, this conventional approach also operates in a future looking way without any retroactive coding configuration.

With the evolution of codes and the need to carefully track and bill the appropriate costs to the appropriate code, there is a need to inspect and retroactively update the codes.

### SUMMARY

The exemplary embodiments disclose a method, a computer program product, and a computer system for retroactively updating a record with a new code. The method comprises determining the new code that is introduced into a medical coding system to which the record is a part. The method comprises determining a first patient having a first patient record, the first patient record including the new code. The method comprises determining a correlation between the new code and an existing code, the existing code being available prior to the new code being introduced. The method comprises determining a second patient having a second patient record, the second patient record including the existing code. The method comprises determining whether the second patient is a candidate to have the second patient record retroactively updated with the new code based on a similarity analysis between the first patient and the second patient. The method comprises as a result of the second patient being confirmed the candidate, updating the second patient record with the new code.

In a preferred embodiment, the new code is determined based on at least one of a user identification of a new code, an outlier, an outcome analysis, a code not recorded in the medical coding system, a new code loaded in the medical coding system, a code not yet seen in the medical coding system, and a code origin comes after a selected date, a triggered load of codes.

In a preferred embodiment, the correlation between the new code and the existing code is determined based on a nearest neighbor analysis with Euclidian distance or density-based scan.

In a preferred embodiment, the similarity analysis is based on demographic data and observation data of the first and second patients, the similarity analysis determining a confidence value of the similarity between the demographic data and the observation data, the confidence value being above a confidence threshold being indicative of a qualified similarity.

In a preferred embodiment, the new code is a set of new codes, the set of new codes being related or unrelated to one another.

In a preferred embodiment, the new code includes one or more extensions.

In a preferred embodiment, the medical coding system is associated with an electronic health record (EHR) system, an electronic medical record (EMR) system, a Fast Healthcare Interoperability Resources (FHIR) server, or a healthcare data store.

### BRIEF DESCRIPTION OF THE SEVERAL VIEWS OF THE DRAWINGS

The following detailed description, given by way of example and not intended to limit the exemplary embodiments solely thereto, will best be appreciated in conjunction with the accompanying drawings, in which:

FIG. 1 depicts an exemplary schematic diagram of a retroactive code system 100, in accordance with the exemplary embodiments.

FIG. 2 depicts an exemplary flowchart of a method 200 illustrating the operations of a retroactive server 130 of the retroactive code system 100 in retroactively updating a record with a new code, in accordance with the exemplary embodiments.

FIG. 3 depicts an exemplary block diagram depicting the hardware components of the retroactive code system 100 of FIG. 1, in accordance with the exemplary embodiments.

FIG. 4 depicts a cloud computing environment, in accordance with the exemplary embodiments.

FIG. 5 depicts abstraction model layers, in accordance with the exemplary embodiments.

The drawings are not necessarily to scale. The drawings are merely schematic representations, not intended to portray specific parameters of the exemplary embodiments. The drawings are intended to depict only typical exemplary embodiments. In the drawings, like numbering represents like elements.

### DETAILED DESCRIPTION OF THE SEVERAL VIEWS OF THE DRAWINGS

Detailed embodiments of the claimed structures and methods are disclosed herein; however, it can be understood that the disclosed embodiments are merely illustrative of the claimed structures and methods that may be embodied in various forms. The exemplary embodiments are only illustrative and may, however, be embodied in many different forms and should not be construed as limited to the exemplary embodiments set forth herein. Rather, these exemplary embodiments are provided so that this disclosure will be thorough and complete, and will fully convey the scope to be covered by the exemplary embodiments to those skilled in the art. In the description, details of well-known features and techniques may be omitted to avoid unnecessarily obscuring the presented embodiments.

References in the specification to “one embodiment”, “an embodiment”, “an exemplary embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to implement such feature, structure, or characteristic in connection with other embodiments whether or not explicitly described.

In the interest of not obscuring the presentation of the exemplary embodiments, in the following detailed description, some processing steps or operations that are known in the art may have been combined together for presentation and for illustration purposes and in some instances may have not been described in detail. In other instances, some processing steps or operations that are known in the art may not be described at all. It should be understood that the following description is focused on the distinctive features or elements according to the various exemplary embodiments.

The exemplary embodiments are directed to a method, computer program product, and system for retroactively updating a record with a new code. The exemplary embodiments provide a mechanism that detects when a new code is introduced and retroactively updates historical medical records or other records with the new code. By identifying first patients who have records that utilize the new code and identifying second patients who may be clustered with the first patients through a similarity analysis, the exemplary embodiments may determine whether the second patients are candidates to retroactively update the records with the new code. Key benefits of the exemplary embodiments may include accurately updating historical records for subsequent operations to be performed correctly, improve identification of missed billing candidates, recover revenue and correct billing for medical institutions, and address complicated problems in pandemic, epidemic, and emerging medical issues with regard to medical data. Detailed implementation of the exemplary embodiments follows.

The exemplary embodiments are described with particular reference to code systems related to managing medical data and associated data (e.g., billing). However, the use of the code system for medical data is only exemplary. The exemplary embodiments may be utilized in any environment in which codes are used to represent an item and historical records may be updated with new codes as they are introduced based on a similarity and/or clustering analysis with first items utilizing the new code and second items that are substantially similar to the first items that do not use the new code.

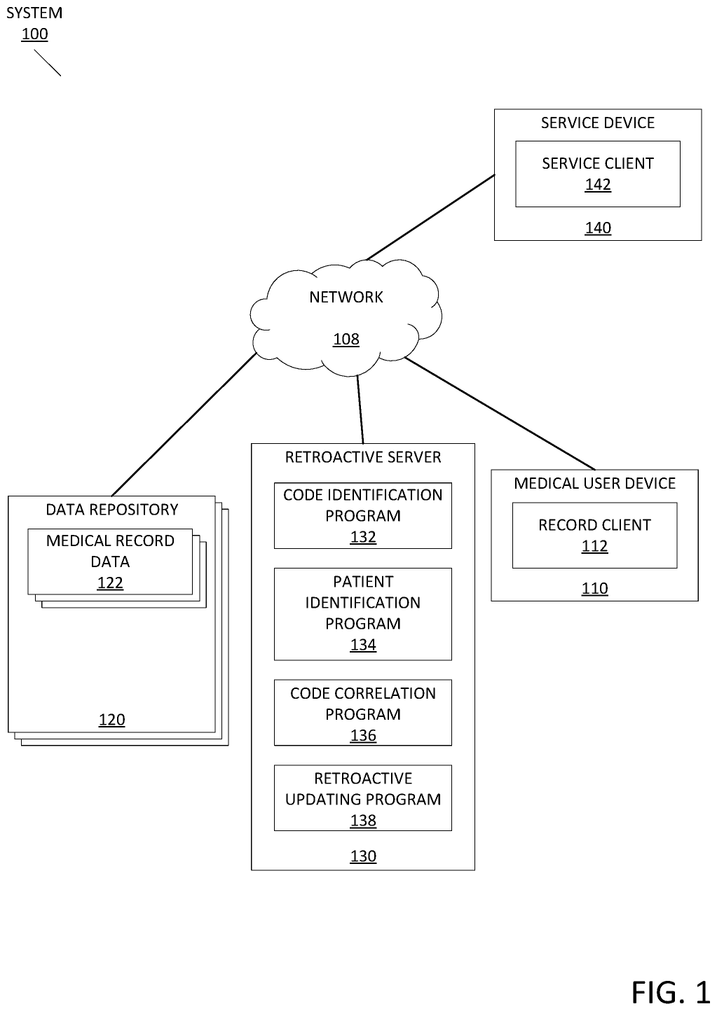

FIG. 1 depicts a retroactive code system 100, in accordance with the exemplary embodiments. According to the exemplary embodiments, the retroactive code system 100 may include a medical user device 110, one or more data repositories 120, a retroactive server 130, and a service device 140, which may all be interconnected via a network 108. While programming and data of the exemplary embodiments may be stored and accessed remotely across several servers via the network 108, programming and data of the exemplary embodiments may alternatively or additionally be stored locally on as few as one physical computing device or amongst other computing devices than those depicted.

In the exemplary embodiments, the network 108 may be a communication channel capable of transferring data between connected devices. Accordingly, the components of the retroactive code system 100 may represent network components or network devices interconnected via the network 108. In the exemplary embodiments, the network 108 may be the Internet, representing a worldwide collection of networks and gateways to support communications between devices connected to the Internet. Moreover, the network 108 may utilize various types of connections such as wired, wireless, fiber optic, etc. which may be implemented as an intranet network, a local area network (LAN), a wide area network (WAN), or a combination thereof. In further embodiments, the network 108 may be a Bluetooth network, a WiFi network, or a combination thereof. In yet further embodiments, the network 108 may be a telecommunications network used to facilitate telephone calls between two or more parties comprising a landline network, a wireless network, a closed network, a satellite network, or a combination thereof. In general, the network 108 may represent any combination of connections and protocols that will support communications between connected devices. For example, the network 108 may also represent direct or indirect wired or wireless connections between the components of the retroactive code system 100 that do not utilize the network 108.

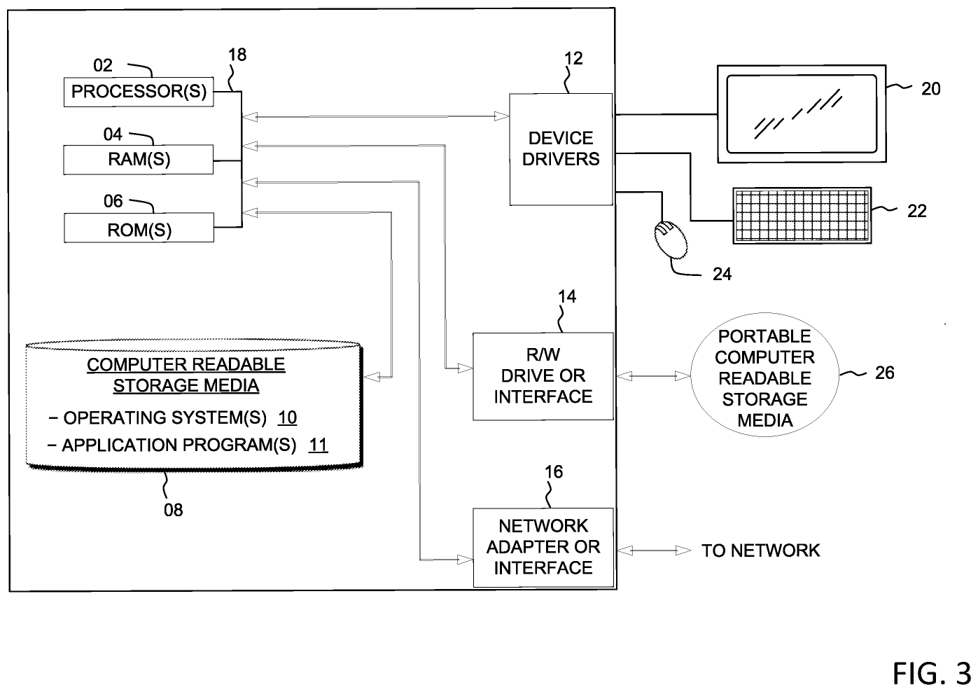

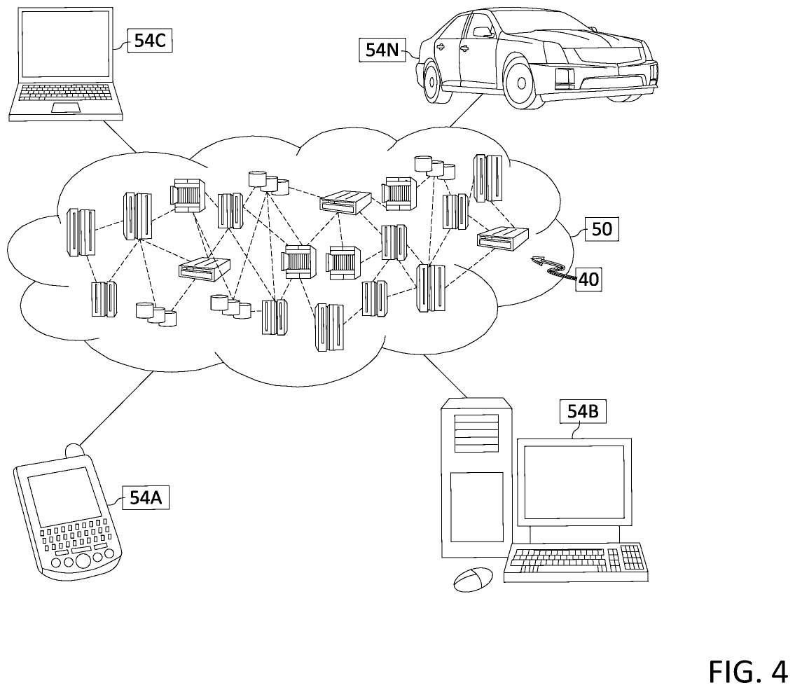

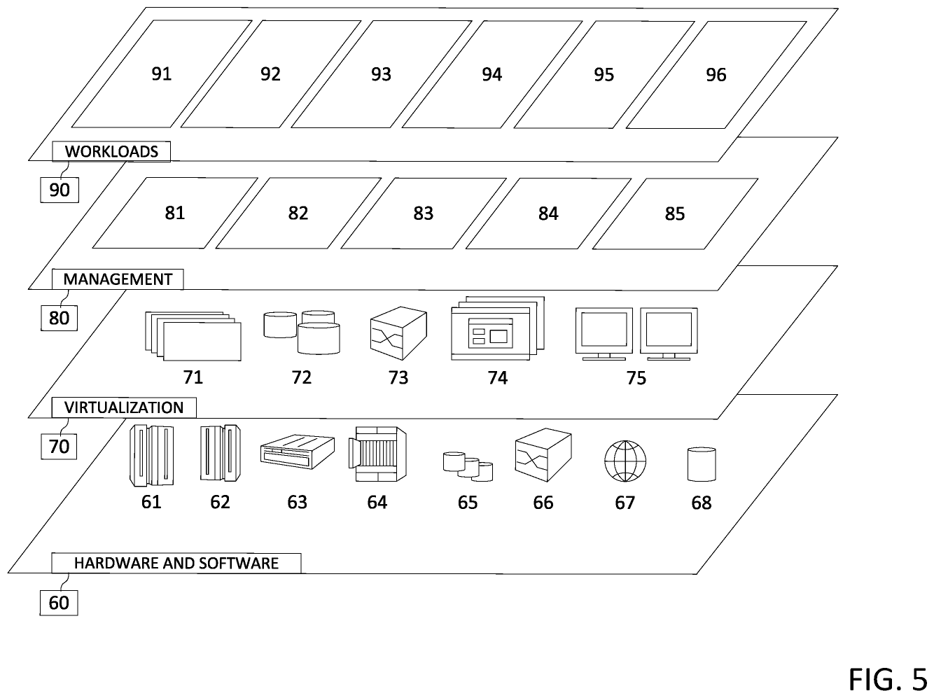

In the exemplary embodiments, the medical user device 110 may include a record client 112, and may be an enterprise server, a laptop computer, a notebook, a tablet computer, a netbook computer, a personal computer (PC), a desktop computer, a server, a personal digital assistant (PDA), a rotary phone, a touchtone phone, a smart phone, a mobile phone, a virtual device, a thin client, an Internet of Things (IoT) device, or any other electronic device or computing system capable of receiving and sending data to and from other computing devices. While the medical user device 110 is shown as a single device, in other embodiments, the medical user device 110 may be comprised of a cluster or plurality of computing devices, in a modular manner, etc., working together or working independently. The medical user device 110 is described in greater detail as a hardware implementation with reference to FIG. 3 (e.g., data processing according to the exemplary embodiments being performed by processor 02), as part of a cloud implementation with reference to FIG. 4 (e.g., the device 110 according to the exemplary embodiments being represented by the laptop computer 54C), and/or as utilizing functional abstraction layers for processing with reference to FIG. 5 (e.g., workload layer 90 including retroactive processing 96 according to the exemplary embodiments). The medical user device 110 may be utilized by a medical professional who enters information about a patient to update a record of that patient.

In the exemplary embodiments, the record client 112 may act as a client in a client-server relationship and may be a software, hardware, and/or firmware based application capable of updating a record of a patient via the network 108. In embodiments, the record client 112 may provide a user interface in which the medical professional may enter a plurality of different types of information from demographics to medically related information as well as interact with one or more components of the retroactive code system 100, and utilize various wired and/or wireless connection protocols for data transmission and exchange associated with data used for modifying a version of an application, including Bluetooth, 2.4 gHz and 5 gHz internet, near-field communication, Z-Wave, Zigbee, etc.

The record client 112 may be any record managing application that enables a medical professional to enter information related to a patient. The medical professional may input general information about the patient such as demographic information (e.g., age, family history, etc.). The medical professional may also input location information such as where the patient resides or has visited (e.g., a disease may be located in a particular geographic area which may be pertinent to a subsequent analysis). The medical professional may further input medical information that are measured as well as results of tests, labs, etc. In inputting medical information, the medical professional may determine a code that corresponds to a medical aspect (e.g., disease or condition) of the patient. When the medical professional cannot definitively identify the medical aspect, the medical professional may utilize a best judgment to select a code. The medical professional may also input a code such as “unknown” or “unidentified” or may select to input a code corresponding to the general procedure performed along with the results. In this manner, the record client 112 may be used to update and manage medical records of patients.

In the exemplary embodiments, the data repository 120 may include one or more medical record data 122 and may be an enterprise server, a laptop computer, a notebook, a tablet computer, a netbook computer, a PC, a desktop computer, a server, a PDA, a rotary phone, a touchtone phone, a smart phone, a mobile phone, a virtual device, a thin client, an IoT device, or any other electronic device or computing system capable of storing, receiving, and sending data to and from other computing devices. While the data repository 120 is shown as a single device, in other embodiments, the data repository 120 may be comprised of a cluster or plurality of electronic devices, in a modular manner, etc., working together or working independently. While the data repository 120 is also shown as a separate component, in other embodiments, the data repository 120 may be incorporated with one or more of the other components of the retroactive code system 100. For example, the data repository 120 may be incorporated in the retroactive server 130. Thus, access to the data repository 120 by the retroactive server 130 may be performed locally. The data repository 120 is described in greater detail as a hardware implementation with reference to FIG. 3, as part of a cloud implementation with reference to FIG. 4, and/or as utilizing functional abstraction layers for processing with reference to FIG. 5.

In the exemplary embodiments, the medical record data 122 may include medical records in various formats such as electronic health records (EHR), electronic medical records (EMR), Fast Healthcare Interoperatbility Resources (FHIR), etc. for respective patients. In this manner, the retroactive code system 100 may be or be part of an EHR system, an EMR system, a FHIR server, a healthcare data store on various platforms, etc. The medical records may include any of the above noted types of information that the medical professional may input via the record client 112. Accordingly, the medical records may electronically store information regarding a patient's health and medical history (e.g., a patient's chart). The patient's health may reflect a most current set of details regarding an overall well-being and/or specific items related to the patient. For example, the patient's health may indicate any conditions, diseases, etc. for which the patient has been diagnosed and whether the condition, disease, etc. has been treated or is ongoing. The medical history may relate to physician visits, results of tests, surgeries performed, treatment plans, etc. The medical information may include other information of the patient such as demographics, medical/family history, medications that were taken or are being taken, allergies, immunization status, radiology images, age, weight, height, etc. as well as non-health related information such as insurance carrier, billing information, etc.

In the exemplary embodiments, the service device 140 may include a service client 142, and may be an enterprise server, a laptop computer, a notebook, a tablet computer, a netbook computer, a personal computer (PC), a desktop computer, a server, a personal digital assistant (PDA), a rotary phone, a touchtone phone, a smart phone, a mobile phone, a virtual device, a thin client, an Internet of Things (IoT) device, or any other electronic device or computing system capable of receiving and sending data to and from other computing devices. While the service device 140 is shown as a single device, in other embodiments, the service device 140 may be comprised of a cluster or plurality of computing devices, in a modular manner, etc., working together or working independently. The service device 140 is described in greater detail as a hardware implementation with reference to FIG. 3 (e.g., data processing according to the exemplary embodiments being performed by processor 02), as part of a cloud implementation with reference to FIG. 4 (e.g., the device 140 according to the exemplary embodiments being represented by the laptop computer 54C), and/or as utilizing functional abstraction layers for processing with reference to FIG. 5 (e.g., workload layer 90 including retroactive processing 96 according to the exemplary embodiments). The service device 140 may be utilized by a service user providing a related service that utilizes the medical record, and particularly any code used in the medical record.

In the exemplary embodiments, the service client 142 may act as a client in a client-server relationship and may be a software, hardware, and/or firmware based application capable of processing a service for a patient through information determined from the patient's medical record via the network 108. In embodiments, the service client 142 may provide a user interface in which the service user may view the medical record of the patient and input information identified therein to process the service for the patient as well as interact with one or more components of the retroactive code system 100, and utilize various wired and/or wireless connection protocols for data transmission and exchange associated with data used for modifying a version of an application, including Bluetooth, 2.4 gHz and 5 gHz internet, near-field communication, Z-Wave, Zigbee, etc.

The service client 142 may be any service application that enables the service user to process information to render the service for the patient. For example, the service provided to the patient may be a billing service. The billing service utilizes the medical services that were provided to the patient (e.g., by the medical professional) as indicated in the medical record and codes indicative of the medical services. The service user may input the codes to determine how to bill the patient for medical services rendered.

The service device 140 including the service client 142 being used by a billing service provider is only for illustrative purposes. The service device 140 may be used by any service user who may incorporate the codes included in the medical record to render a subsequent service. For example, in another exemplary embodiment, the service device 140 may be part of a recommendation provider who ingests the codes and outputs a recommended course of medical treatment. In this manner, the billing service described above and used herein may represent any service that is provided to the patient in which the codes of the medical record may be incorporated.

In the exemplary embodiments, the retroactive server 130 may include a code identification program 132, a patient identification program 134, a code correlation program 136, and a retroactive updating program 138, and act as a server in a client-server relationship with the record client 112 and the service client 142 as well as be in a communicative relationship with the data repository 120. The retroactive server 130 may be an enterprise server, a laptop computer, a notebook, a tablet computer, a netbook computer, a PC, a desktop computer, a server, a PDA, a rotary phone, a touchtone phone, a smart phone, a mobile phone, a virtual device, a thin client, an IoT device, or any other electronic device or computing system capable of receiving and sending data to and from other computing devices. While the retroactive server 130 is shown as a single device, in other embodiments, the retroactive server 130 may be comprised of a cluster or plurality of computing devices, working together or working independently. The retroactive server 130 is described in greater detail as a hardware implementation with reference to FIG. 3 (e.g., data processing according to the exemplary embodiments being performed by processor 02), as part of a cloud implementation with reference to FIG. 4 (e.g., the device 110 according to the exemplary embodiments being represented by the desktop computer 54B), and/or as utilizing functional abstraction layers for processing with reference to FIG. 5 (e.g., workload layer 90 including retroactive processing 96 according to the exemplary embodiments).

In the exemplary embodiments, the code identification program 132 may be a software, hardware, and/or firmware application configured to determine a code that is used in medical records. The code identification program 132 may also be configured to determine when a new code is introduced into the medical code system to which the medical record is a part. In the exemplary embodiments, the patient identification program 134 may be a software, hardware, and/or firmware application configured to determine a patient who have select characteristics. In this manner, the patient identification program 134 may determine, for example, a patient whose medical record is utilizing a new code as well as a patient whose medical record is not utilizing a new code. The patient identification program 134 may further determine how patients may be clustered (e.g., in a cohort) or otherwise determine how patients are similar to one another based on various factors (e.g., demographics, medical history, conditions, diseases, etc.). The patient identification program 134 may utilize demographic data and observation data between patients and generate a confidence value such that the confidence value being above a confidence threshold is indicative of a qualified similarity of the patients. In the exemplary embodiments, the code correlation program 136 may be a software, hardware, and/or firmware application configured to determine how a new code is correlated to one or more existing codes. As will be described in further detail below, a medical professional may have examined a patient and entered a code for a condition that does not yet have a specific code and therefore one or more existing codes are used in its stead. At a subsequent time, the new code for the condition may be introduced at which time the code correlation program 136 may determine how the new code correlates to one or more existing codes. In the exemplary embodiments, the retroactive updating program 138 may be a software, hardware, and/or firmware application configured to utilize the results and information from the code identification program 132, the patient identification program 134, and the code correlation program 136 to determine a patient who is a candidate for a new code to be retroactively updated in a respective medical record. The retroactive updating program 138 may be configured to provide this feature as a recommendation (e.g., prompt the medical professional who entered the existing code and request confirmation of updating with the new code) or automatically (e.g., perform a confidence analysis where a confidence value being above a predetermined threshold is indicative of the existing code to be retroactively updated with the new code).

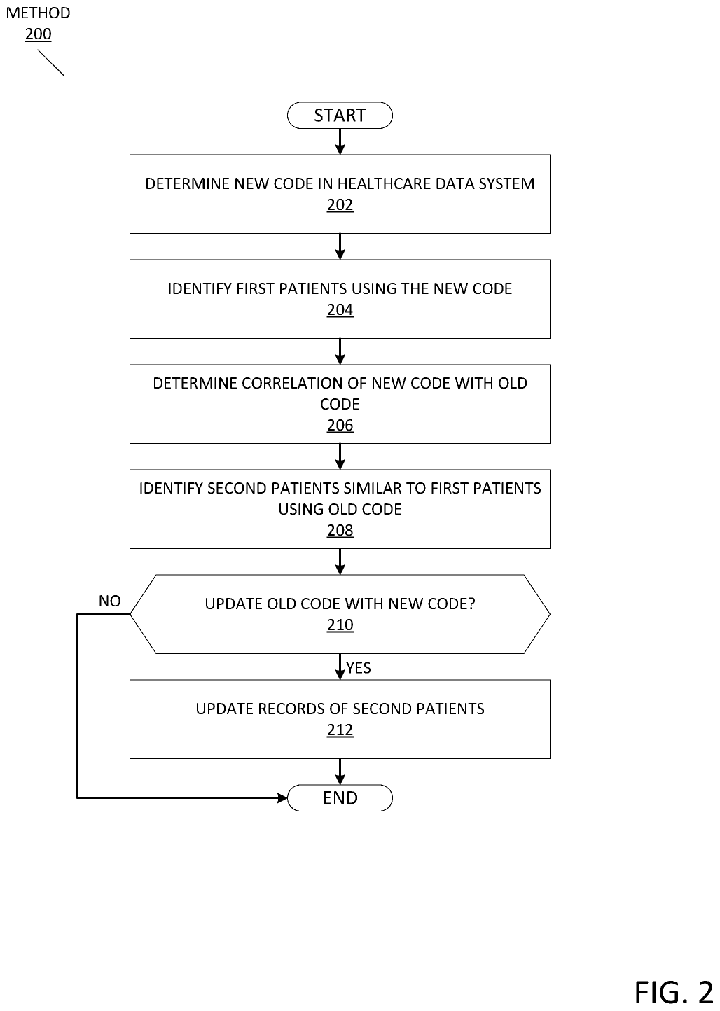

FIG. 2 depicts an exemplary flowchart of a method 200 illustrating the operations of the retroactive server 130 of the retroactive code system 100 in retroactively updating a record with a new code, in accordance with the exemplary embodiments. The method 200 may relate to operations that are performed by the code identification program 132, the patient identification program 134, the code correlation program 136, and the retroactive updating program 138. The method 200 will be described from the perspective of the retroactive server 130.

The retroactive server 130 may determine a new code that is introduced in the medical coding system to which a medical record may be a part (step 202). The retroactive server 130 may be configured to determine the introduction of the new code using a variety of different mechanisms, either alone or as a combination. In a first example, the retroactive server 130 may utilize a user identification of the new code where a skilled administrator may select the new code and start date for the new code. In a second example, the retroactive server 130 may identify the new code as an outlier where a selected code is new or significantly unique, such as being outside of two standard deviations. In a third example, the retroactive server 130 may utilize an outcome analysis by determining a change in outcomes, and then assigning the changed outcome for a demographic to a candidate code. In a fourth example, the retroactive server 130 may determine a selected code that is not recorded in medical coding system where the new code is used or referenced in the medical coding system. In a fifth example, the retroactive server 130 may determine the new code that is now loaded in medical coding system or the new code is not seen in medical coding system where the new code may not be loaded or stored in a lookup table of the medical coding system. In a sixth example, the retroactive server 130 may determine when a selected code has an origin that occurs after a particular date such as when the origin date is new in the medical coding system. In a seventh example, the retroactive server 130 may utilize a triggered load of codes where after a new code is loaded, an asynchronous analysis may be triggered. The retroactive server 130 may utilize any other mechanism to determine the new code as one skilled in the art will recognize. In this manner, the retroactive server 130 may utilize any one or more of the above mechanisms to determine the new code.

The retroactive server 130 may also process the new code in a singular manner or as a set of new codes. For example, the determined new code may be a set of new codes that may be related or even unrelated to one another. In a particular exemplary embodiment, the determined new code may have a start date with an explicit relevance date (e.g., COVID-19 only goes back a certain number of months relative to a current time). The determined new code may contain extensions to existing codes to establish a set of new codes (e.g., U07.1, Bronchial, Pneumonia, COVID-19).

As described above, the medical coding system may be part of a healthcare data system that integrates with an EHR system, an EMR system, a FHIR Server, a healthcare data store, etc. Each of the encounters, observations, and diagnostic reports may be sent as a resource or queue of HL7v2 messages to an analytical system (e.g., via the record client 112). The analytical portion of the retroactive code system 100 may enter the data into a data table where the data is extracted from the standard format into an analytical format. The analytical format may contain patient and demographic data (e.g., age, location, observation, and codes). Based on the medical aspect to which the new code or the set of new codes pertains, the retroactive server 130 may limit the analysis to a specific time such as a start of an outbreak, or at most a predetermined amount of time prior to a current time (e.g., six months).

The retroactive server 130 may identify one or more first patients using the new code in respective medical records (step 204). For example, there may be three medical records where each medical record corresponds to a first patient, a second patient, and a third patient, the three patients each belonging to a common demographic (e.g., age range). The first and third patients may include the new code (e.g., U07.1) while the second patient includes an existing code (e.g., X7.9). In this manner, the retroactive server 130 may identify the first and third patients as a basis for subsequent processing.

The retroactive server 130 may determine a correlation between the new code and one or more existing codes (step 206). The retroactive server 130 may utilize a variety of different techniques in determining correlations between the new code and the existing codes. For example, the retroactive server 130 may use a nearest neighbor (e.g., kNN) with Euclidian distance or density-based scan to determine outliers which need to associate with a closer. The retroactive server 130 may also group patients into cohorts based on codes associated with demographics. The retroactive server 130 may further associate the data with the code based on the similar features. In this manner, the retroactive server 130 may associate or correlate the new code with existing codes based on the similarity of the patients with the new codes and evaluates based on the similarity of the likelihood for an update, as will be described in further detail below. The retroactive server 130 may utilize further analyses to determine a correlation between a new code and one or more existing codes including those that one skilled in the art will understand (e.g., machine learning techniques, neural networks including convolution/recurrent/artificial, etc.).

The retroactive server 130 may identify second patients similar to the first patients using the one or more existing codes correlated to the new code (step 208). Using the patient demographic and observation data associated with the determined existing code correlated to the new code, the retroactive server 130 may run the prior data (e.g., the medical records of patients) that does not associate or use the new code to confirm the second patients.

The retroactive server 130 may determine whether any of the second patients qualifies to have the one or more existing codes retroactively updated with the new code (decision 210). For each of the second patients who are identified candidates for the retroactive updating of the respective medical record, the retroactive server 130 determines how similar the demographics and the observation data are to the new code for each second patient. If the new code is within a certain percentage (e.g., similarity threshold), the retroactive server 130 may tag this second patient as a possible recoding candidate. In this manner, the retroactive server 130 may determine whether a specific one of the second patients qualifies for the retroactive updating. As a result of the second patient not qualifying (decision 210, “NO” branch), the retroactive server 130 may retain the use of the one or more existing codes in the medical record for the second patient. As a result of the second patient qualifying (decision 210, “YES” branch), the retroactive server 130 may update the medical record of the second patient to replace the one or more existing codes with the new code or otherwise annotate that the one or more existing codes correlate to the new code (step 212).

The retroactive server 130 may retroactively label or update a code based on a wave or a phase or mutations. For example, strain-1 or strain-2 in a cohort group is used to label a candidate for recoding. The identified retroactive coding may create candidate FHIR to update the coding and replace the existing coded resources. The retroactive server 130 may also be configured to update the medical records or resources as well as forward this update downstream, for example, to a billing system.

The retroactive server 130 may further be configured with various security features. For example, the retroactive server 130 may analyze the usage of the new code to ensure that proprietary or mandated guidelines are followed (e.g., HIPAA, GDPR, etc.). In another example, the retroactive server 130 may conduct a security scan to ensure that the process of using a new code does not introduce any malevolent attacks to the retroactive code system (e.g., hacking opportunities).

To further illustrate the operations of the retroactive server 130, reference is now made to an illustrative exemplary process. According to the illustrative exemplary process, a patient F may be a patient with a medical network. The patient F has been admitted to the hospital and had an encounter with the physician's assistant (PA). The PA examines the patient F and determines a potential case of viral pneumonia, not one that has been seen before in the patient. The subjective and objective data is entered into the EMR of the patient F. The PA fills in a diagnostic report and an observation with code J12.89, “Other viral pneumonia” and code B97.29, “Other coronavirus as the cause of diseases classified elsewhere.” The PA sends the EMR on to the appropriate healthcare workflow. The workflow generates claims, bills, prescriptions, observations and diagnostic reports. At a subsequent time, the WHO introduces a new classification for the observation or case that the PA described. Accordingly, the retroactive server 130 may detects a new code U07.1, “Bronchial, Pneumonia, COVID-19” loaded to the medical coding system. This new code was not previously used in the medical coding system and is recently added, where the patient F was examined prior to the introduction of the new code U07.1. The retroactive server 130 may select the patients and patients' healthcare data that uses the new code. For example, patient S in a first age group includes the new code U07.1 with an extension of “Bronchial”. The patient S may have been seen one week after loading the new code into the medical coding system. In another example, patient A in a second age group different than the first age group includes the new code with an extension of “Asthma Signs.” The patient A may have been seen three days after loading the new code into the medical coding system. The retroactive server 130 may identify the association or correlation between the new code and demographics as well as the association or correlation between the observation and diagnostics. The retroactive server 130 may determine a similarity of the patient F to the patient S and the patient A. Accordingly, the retroactive server 130 may identify that the code for the patient F is a candidate for retroactive updating. For example, the retroactive updating may result in updating a billing to reduce or reassign associated costs for the patient F as the previously used existing codes (e.g., J12,89 and/or B97.29 may have higher associated costs). According to an exemplary implementation, the retroactive server 130 may determine that the patient F is a candidate for the retroactive updating and provides a recommendation to an administrator of the medical record for the patient F. The administrator may log into the system and accept the recommendation to reclassify the patient F's case. The patient F's claim may be refiled and appropriately billed and reduced, and the dashboard may be updated with actual statistics related to the diseases under study corresponding to the new code.

The exemplary embodiments are configured to retroactively update a medical record with a new code. In detecting the introduction of a new code into a medical coding system, the exemplary embodiments may determine first patients with medical records that include the new code. The exemplary embodiments may determine a correlation of the new code with existing codes, particular for medical records that are maintained prior to the introduction of the new code. The exemplary embodiments may determine second patients having the medical records that do not include the new code who are substantially similar to the first patients. In this manner, the exemplary embodiments may determine candidates among the second patients who are to have corresponding medical records retroactively updated with the new code.

FIG. 3 depicts a block diagram of devices within the retroactive code system 100 of FIG. 1, in accordance with the exemplary embodiments. It should be appreciated that FIG. 3 provides only an illustration of one implementation and does not imply any limitations with regard to the environments in which different embodiments may be implemented. Many modifications to the depicted environment may be made.

Devices used herein may include one or more processors 02, one or more computer-readable RAMs 04, one or more computer-readable ROMs 06, one or more computer readable storage media 08, device drivers 12, read/write drive or interface 14, network adapter or interface 16, all interconnected over a communications fabric 18. Communications fabric 18 may be implemented with any architecture designed for passing data and/or control information between processors (such as microprocessors, communications and network processors, etc.), system memory, peripheral devices, and any other hardware components within a system.

One or more operating systems 10, and one or more application programs 11 are stored on one or more of the computer readable storage media 08 for execution by one or more of the processors 02 via one or more of the respective RAMs 04 (which typically include cache memory). In the illustrated embodiment, each of the computer readable storage media 08 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

Devices used herein may also include a RAY drive or interface 14 to read from and write to one or more portable computer readable storage media 26. Application programs 11 on said devices may be stored on one or more of the portable computer readable storage media 26, read via the respective R/W drive or interface 14 and loaded into the respective computer readable storage media 08.

Devices used herein may also include a network adapter or interface 16, such as a TCP/IP adapter card or wireless communication adapter (such as a 4G wireless communication adapter using OFDMA technology). Application programs 11 on said computing devices may be downloaded to the computing device from an external computer or external storage device via a network (for example, the Internet, a local area network or other wide area network or wireless network) and network adapter or interface 16. From the network adapter or interface 16, the programs may be loaded onto computer readable storage media 08. The network may comprise copper wires, optical fibers, wireless transmission, routers, firewalls, switches, gateway computers and/or edge servers.

Devices used herein may also include a display screen 20, a keyboard or keypad 22, and a computer mouse or touchpad 24. Device drivers 12 interface to display screen 20 for imaging, to keyboard or keypad 22, to computer mouse or touchpad 24, and/or to display screen 20 for pressure sensing of alphanumeric character entry and user selections. The device drivers 12, RAY drive or interface 14 and network adapter or interface 16 may comprise hardware and software (stored on computer readable storage media 08 and/or ROM 06).

The programs described herein are identified based upon the application for which they are implemented in a specific one of the exemplary embodiments. However, it should be appreciated that any particular program nomenclature herein is used merely for convenience, and thus the exemplary embodiments should not be limited to use solely in any specific application identified and/or implied by such nomenclature.

Based on the foregoing, a computer system, method, and computer program product have been disclosed. However, numerous modifications and substitutions can be made without deviating from the scope of the exemplary embodiments. Therefore, the exemplary embodiments have been disclosed by way of example and not limitation.

It is to be understood that although this disclosure includes a detailed description on cloud computing, implementation of the teachings recited herein are not limited to a cloud computing environment. Rather, the exemplary embodiments are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 4, illustrative cloud computing environment 50 is depicted. As shown, cloud computing environment 50 includes one or more cloud computing nodes 40 with which local computing devices used by cloud consumers, such as, for example, personal digital assistant (PDA) or cellular telephone 54A, desktop computer 54B, laptop computer 54C, and/or automobile computer system 54N may communicate. Nodes 40 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 40 and cloud computing environment 50 can communicate with any type of computerized device over any type of network and/or network addressable connection (e.g., using a web browser).

Referring now to FIG. 5, a set of functional abstraction layers provided by cloud computing environment 50 (FIG. 4) is shown. It should be understood in advance that the components, layers, and functions shown in FIG. 5 are intended to be illustrative only and the exemplary embodiments are not limited thereto. As depicted, the following layers and corresponding functions are provided:

Hardware and software layer 60 include hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and retroactive processing 96.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

1. A computer-implemented method for retroactively updating a record with a new code, the method comprising:
determining the new code that is introduced into a medical coding system to which the record is a part;
determining a first patient having a first patient record, the first patient record including the new code;
determining a correlation between the new code and an existing code, the existing code being available prior to the new code being introduced;
determining a second patient having a second patient record, the second patient record including the existing code;
determining whether the second patient is a candidate to have the second patient record retroactively updated with the new code based on a similarity analysis between the first patient and the second patient; and
as a result of the second patient being confirmed the candidate, updating the second patient record with the new code.

2. The computer-implemented method of claim 1, wherein the new code is determined based on at least one of a user identification of a new code, an outlier, an outcome analysis, a code not recorded in the medical coding system, a new code loaded in the medical coding system, a code not yet seen in the medical coding system, and a code origin comes after a selected date, a triggered load of codes.

3. The computer-implemented method of claim 1, wherein the correlation between the new code and the existing code is determined based on a nearest neighbor analysis with Euclidian distance or density-based scan.

4. The computer-implemented method of claim 1, wherein the similarity analysis is based on demographic data and observation data of the first and second patients, the similarity analysis determining a confidence value of the similarity between the demographic data and the observation data, the confidence value being above a confidence threshold being indicative of a qualified similarity.

5. The computer-implemented method of claim 1, wherein the new code is a set of new codes, the set of new codes being related or unrelated to one another.

6. The computer-implemented method of claim 1, wherein the new code includes one or more extensions.

7. The computer-implemented method of claim 1, wherein the medical coding system is associated with an electronic health record (EHR) system, an electronic medical record (EMR) system, a Fast Healthcare Interoperability Resources (FHIR) server, or a healthcare data store.

8. A non-transitory computer-readable storage media that configures a computer to perform program instructions stored on the non-transitory computer-readable storage media for retroactively updating a record with a new code, the program instructions comprising:
determining the new code that is introduced into a medical coding system to which the record is a part;
determining a first patient having a first patient record, the first patient record including the new code;
determining a correlation between the new code and an existing code, the existing code being available prior to the new code being introduced;
determining a second patient having a second patient record, the second patient record including the existing code;
determining whether the second patient is a candidate to have the second patient record retroactively updated with the new code based on a similarity analysis between the first patient and the second patient; and
as a result of the second patient being confirmed the candidate, updating the second patient record with the new code.

9. The computer program product of claim 8, wherein the new code is determined based on at least one of a user identification of a new code, an outlier, an outcome analysis, a code not recorded in the medical coding system, a new code loaded in the medical coding system, a code not yet seen in the medical coding system, and a code origin comes after a selected date, a triggered load of codes.

10. The computer program product of claim 8, wherein the correlation between the new code and the existing code is determined based on a nearest neighbor analysis with Euclidian distance or density-based scan.

11. The computer program product of claim 8, wherein the similarity analysis is based on demographic data and observation data of the first and second patients, the similarity analysis determining a confidence value of the similarity between the demographic data and the observation data, the confidence value being above a confidence threshold being indicative of a qualified similarity.

12. The computer program product of claim 8, wherein the new code is a set of new codes, the set of new codes being related or unrelated to one another.

13. The computer program product of claim 8, wherein the new code includes one or more extensions.

14. The computer program product of claim 8, wherein the medical coding system is associated with an electronic health record (EHR) system, an electronic medical record (EMR) system, a Fast Healthcare Interoperability Resources (FHIR) server, or a healthcare data store.

15. A computer system for retroactively updating a record with a new code, the computer system comprising:
one or more computer processors, one or more computer-readable storage media, and program instructions stored on the one or more of the computer-readable storage media for execution by at least one of the one or more processors capable of performing a method, the method comprising:
determining the new code that is introduced into a medical coding system to which the record is a part;
determining a first patient having a first patient record, the first patient record including the new code;
determining a correlation between the new code and an existing code, the existing code being available prior to the new code being introduced;
determining a second patient having a second patient record, the second patient record including the existing code;
determining whether the second patient is a candidate to have the second patient record retroactively updated with the new code based on a similarity analysis between the first patient and the second patient; and
as a result of the second patient being confirmed the candidate, updating the second patient record with the new code.

16. The computer system of claim 15, wherein the new code is determined based on at least one of a user identification of a new code, an outlier, an outcome analysis, a code not recorded in the medical coding system, a new code loaded in the medical coding system, a code not yet seen in the medical coding system, and a code origin comes after a selected date, a triggered load of codes.

17. The computer system of claim 15, wherein the correlation between the new code and the existing code is determined based on a nearest neighbor analysis with Euclidian distance or density-based scan.

18. The computer system of claim 15, wherein the similarity analysis is based on demographic data and observation data of the first and second patients, the similarity analysis determining a confidence value of the similarity between the demographic data and the observation data, the confidence value being above a confidence threshold being indicative of a qualified similarity.

19. The computer system of claim 15, wherein the new code is a set of new codes, the set of new codes being related or unrelated to one another.

20. The computer system of claim 15, wherein the new code includes one or more extensions.
